(19) United States
(12) Patent Application Publication
(10) Pub. No.: US 2010/0212666 A1
(43) Pub. Date: Aug. 26, 2010

(54) System for Controlling Administration of Anaesthesia

(75) Inventors: Thomas Bouillon, Basel (CH); Antonello Caruso, Zurich (CH); Martin Luginbühl, Hinterkappelen (CH); Manfred Morari, Kusnacht (CH); Peter Matthias Schumacher, Kirchlindach (CH); Eleonora Zanderigo, London (GB)

Correspondence Address: SHOEMAKER AND MATTARE, LTD, 10 POST OFFICE ROAD - SUITE 100, SILVER SPRING, MD, 20910, US

(73) Assignees: UNIVERSITAT BERN, Bern (CH); ETH TECHNOLOGIETRANSFER, Zurich (CH)

(21) Appl. No.: 12/305,806

(22) PCT Filed: Jun. 12, 2007
(86) PCT No.: PCT/EP2007/005165

§ 371 Date: Jan. 2, 2009

(30) Foreign Application Priority Data
Jun. 21, 2006 (EP) 06102702.4

(51) Int. Cl. A61M 16/01

(52) U.S. Cl. 128/203.14; 128/203.12

(57) ABSTRACT

A system for controlling administration of a respiratory depressant drug or mixture of drugs to a spontaneously breathing patient comprises a drug delivery unit (3), being adapted for indexed or continuous and automatic titration of a respiratory depressant drug or mixture of such drugs to said patient (1), and a control apparatus (6), receiving measurement signals (20) relative to the respiratory state of said patient (1) and issuing control signals (27) to said drug delivery unit (3), wherein the control apparatus is adapted to keep said measurement signals relative to the respiratory state to a predetermined condition and thereby providing adequate sedation and/or analgesia to said patient.

### BACKGROUND OF THE INVENTION

1. Field of the Invention

The invention relates generally to a system and associated procedures to administer one or more respiratory depressant drugs to a spontaneously breathing patient. More particularly, the invention relates to an arrangement for providing patient sedation and alleviating pain, discomfort and/or anxiety during a medical or surgical treatment. Said arrangement determines and delivers the optimal administration profile based on the monitored and/or inferred respiratory drive, expressed as body contents of respiratory gases and/or breathing activity. The invention enables the delivery of efficacious patient sedation while minimizing drug-induced adverse effects under both open- and closed-loop operation.

2. Description of Related Art

Anaesthesia refers to a condition of reduced sensibility in the body. It is a reversible pharmacologic state induced by the administration of anaesthetic drugs. Delivery of adequate anaesthesia during medical treatments ensures patient unconsciousness, analgesia and/or muscle relaxation.

According to the International Association for the Study of Pain (IASP), regular re-evaluation of drug dosing during medical and surgical treatments is required. Pronounced individual variability in the response to the drug and to surgical stimuli, combined with changes in responsiveness over time, requires the individualization of drug delivery. Tailoring the administration profile to a patient is based on a continuing process of effect appraisal and dose titration. The objective is to optimize the desired effects (e.g. analgesia, anxiolysis and sedation) while minimizing the undesired effects (IASP Task Force, 2005). The present invention relates to a care system and method to effectively address these issues.

Sedation techniques are currently available to provide patient analgesia and anxiolysis during medical and surgical procedures. “Conscious sedation” and “monitored anaesthesia care” (MAC) are defined as medically controlled states of depressed consciousness that allow protective reflexes to be maintained. The sedated patient retains the ability to breathe autonomously and to protect the airways. Depending on the depth of sedation, the patient can respond to verbal commands and tactile stimulation with different degrees of purposefulness (Novak, 1998). During the delivery of conscious sedation, a physician supervises or personally administers sedative and/or analgesic drugs that allay patient anxiety and ensure analgesia throughout a diagnostic or therapeutic procedure. Such pharmacologic depression of consciousness to a moderate level of sedation is intended to facilitate the successful performance of the medical procedure while providing patient comfort, anxiolysis and cooperation. MAC, on the other hand, allows for the safe administration of a maximal depth of sedation in excess of that provided during conscious sedation (ASA Relative Value Guide, 2006).

The use of conscious sedation and MAC is widespread for, but not limited to, the following treatments: endoscopy (such as gastroscopy, colonoscopy, endoscopic retrograde cholangiopancreatography (ERCP)); bronchoscopy or fiber optic intubation; cystoscopy; extracorporeal shockwave lithotripsy (ESWL); evacuation of chronic epidural haematoma (combined with local anaesthesia); debridement of wounds/burns, abscess drainage; virtual probe plastic surgery; interventional radiology; oocyte harvest for artificial fertilization; dental surgical interventions (combined with local anaesthesia); opthalmologic procedures (combined with retrobulbar block).

Several factors make conscious sedation and MAC attractive compared to general anaesthesia. Low drug and equipment costs, the lack of prolonged monitoring following the procedure, the short duration of patient recovery keep clinical costs at a moderate level. Conversely, general anaesthesia and the subsequent post-anaesthesia care have a significant impact on clinical costs in terms of equipment, medications and human resources. Moreover, the induction of a prolonged state of unconsciousness in the patient affects adversely the duration and quality of recovery. Patients treated with conscious sedation and/or MAC are usually de-hospitalized faster than those undergoing general anaesthesia. On the other hand, conscious sedation and MAC are not suitable for all surgical treatments. Markedly invasive surgeries, for example, usually require the provision of general anaesthesia and delay patient home readiness.

Several different drugs can be used to provide patient sedation and, at higher dosage, general anaesthesia. Opioids (such as remifentanil, alfentanil, fentanyl, sufentanil and meperidine), benzodiazepines (as midazolam, diazepam and lorazepam), propofol and ketamine are examples. In the clinical setting, sedatives and analgesics are delivered orally, rectally, intravenously or intramuscularly. Opioids are administered intravenously. Hypnotics are delivered either intravenously or by inhalation in case of volatile agents. Regardless of the administration method, most of these drugs have a marked respiratory depressant effect, which depends on dosage, administration profile, patient sensitivity and health conditions. High dosages and/or fast administration rates can determine a dangerous impairment of the respiratory drive, and ultimately lead to apnoea and death. Moreover respiratory inhibition sets up an acidic state in the body due to the excessive amount of CO2. This phenomenon is referred to as respiratory acidosis (equivalent terms are hypercapnic acidosis and carbon dioxide acidosis). Acute respiratory acidosis can be life-threatening when a sharp increase in PaCO2 is associated with severe hypoxemia and acidemia. Indeed, the American Society of Anesthesiologists (ASA) identifies drug-induced respiratory depression in the spontaneously ventilating patient as a primary cause of morbidity (ASA Task Force, 2006).

From what discussed so far, it is evident that the delivery of sedation to a patient represents a trade-off between pain relief and anxiety control, and respiratory drive inhibition, loss of airway protection and unconsciousness. Therefore it comes as no surprise that drug-induced respiratory depression, blood deoxygenation and respiratory acidosis are the most prominent side effects of sedation. These effects are especially common if two or more classes of drugs are administered concomitantly (Bhananker et al., 2006). Adequate oxygenation is often ensured by providing an extra flow of oxygen to the patient, e.g. via a nasal cannula or a non-rebreather face mask. However, adequate oxygenation does not imply adequate respiration in the non-steady state nor when breathing an oxygen enriched gas mixture.

U.S. Pat. No. 5,806,513 (Tham et al.) discloses a control system, which enables closed-circuit anaesthesia delivery systems to maintain user-defined oxygen and anaesthetic concentrations via flow minimization routines.

U.S. Pat. No. 7,034,692 (Hickle) discloses a system to monitor the ventilatory conditions of a patient and prevent false, annoying or oversensitive alarms during the performance of a medical procedure. Monitoring data are processed by a high-sensitivity alarm algorithm and a high-specificity alarm algorithm, which generate silent, semi-overt, or overt alarm conditions and/or activate a hyper-vigilant state in the system. Said system provides automated responses to the high-sensitivity, high-specificity alarm algorithms and reduces false positive/false negative alarms in a user-transparent way.

P.C.T. Pat. No. WO 2005/082369 (Shafer et al.) discloses an opioid formulation for pulmonary administration in the treatment of pain. The formulation is dispensed by a pulmonary drug delivery device, which can require a deliberate patient effort to be actuated. Said formulation comprises at least one rapid-onset opioid and at least one sustained-effect opioid (for example, an opioid encapsulated in a biocompatible carrier that delays release of the drug at the lung surface, such as a liposome-encapsulated opioid). The formulation employs the side effects of the rapid-onset and liposomally encapsulated opioids to permit patients to self-limit drug intake.

### 3. REFERENCES

M. Akio, N. Akiya, K. Shinobu, K. Yasushi, and K. Yoichiro. Clinical evaluation of a new combined transcutaneous carbon dioxide tension and pulse oximetry sensor in adult patients undergoing laparoscopic surgery. ASA Annual Meeting Abstracts, 2004.
L. C. Novak. ASA updates its position on monitored anesthesia care. American Society of Anesthesiologists Newsletter, 62(12): 1998.
S. M. Bhananker, K. L. Posner, F. W. Cheney, R. A. Caplan, L. A. Lee, and K. B. Domino. Injury and liability associated with monitored anesthesia care. Anesthesiology, 104: 228-234, 2006.
International Association for the Study of Pain. Task force on professional education: Core curriculum for professional education in pain. IASP Press, 2005.
American Society of Anesthesiologists. Task Force on Sedation and Analgesia by Non-anesthesiologists. Anesthesiology, 84(2): 459-471, 1996.
American Society of Anesthesiologists. ASA Statement: Distinguishing Monitored Anesthesia Care (MAC) from Moderate Sedation/Analgesia (Conscious Sedation). ASA 2006 Relative Value Guide, 2006.
E. Sarton, A. Dahan, L. Teppema, A. Berkenbosch, M. Van Den Elsen, J. Van Kleef. Influence of acute pain induced by activation of cutaneous nociceptors on ventilatory control. Anesthesiology, 87(2):289-296, 1997.
C. J. Glynn, J. W. Lloyd, S. Folkhard. Ventilatory response to intractable pain. Pain, 11: 201-211, 1981.
S. Schraag, G. N. Kenny, U. Mohl, M. Georgieff. Patient-maintained remifentanil target-controlled infusion for the transition to early postoperative analgesia. Br. J. Anaesth., 81: 365-368, 1998.
L. I. Cortinez, H. R. Munoz, R. De la Fuente, D. Acuna, J. A. Dagnino. Target-controlled infusion of remifentanil or fentanyl during extra-corporeal shock-wave lithotripsy. Eur. J. Anaesthesiol., 22: 56-61, 2005.
T. Bouillon, J. Bruhn, L. Radu-Radulescu, C. Andresen, C. Cohane, S. L. Shafer. A model of the ventilatory depressant potency of remifentanil in the non-steady state. Anesthesiology, 99: 779-787, 2003.
M. C. van den Nieuwenhuyzen, F. H. Engbers, A. G. Burm, A. A. Vletter, J. W. van Kleef, J. G. Bovill. Target-controlled infusion of alfentanil for postoperative analgesia: a feasibility study and pharmacodynamic evaluation in the early postoperative period. Br. J. Anaesth., 78: 17-23, 1997.
S. Schraag, M. R. Checketts, G. N. Kenny. Lack of rapid development of opioid tolerance during alfentanil and remifentanil infusions for postoperative pain. Anesth. Analg., 89: 753-757, 1999.
T. Bouillon, C. Schmidt, G. Garstka, D. Heimbach, D. Stafforst, H. Schwilden, A. Hoeft. Pharmacokinetic-pharmacodynamic modeling of the respiratory depressant effect of alfentanil. Anesthesiology, 91: 144-155, 1999.
T. Bouillon, J. Bruhn, L. Radulescu, C. Andresen, T. J. Shafer, C. Cohane, S. L. Shafer. Pharmacodynamic interaction between propofol and remifentanil regarding hypnosis, tolerance of laryngoscopy, bispectral index, and electroencephalographic approximate entropy. Anesthesiology, 100: 1353-1372, 2004.
T. Bouillon, J. Bruhn, L. Radu-Radulescu, C. Andresen, C. Cohane, S. L. Shafer. Mixed-effects modeling of the intrinsic ventilatory depressant potency of propofol in the non-steady state. Anesthesiology, 100: 240-250, 2004.
D. R. Westenskow, A. M. Zbinden, D. A. Thomson, B. Kohler. Control of end-tidal halothane concentration. Part A: Anaesthesia breathing system and feedback control of gas delivery. Br. J. Anaesth., 58(5): 555-562, 1986.

### SUMMARY OF THE INVENTION

A wide variety of drugs is used in modern anaesthetic practice. Some of the most common general anaesthetics are barbiturates, benzodiazepines, ketamine and propofol. Opioids, on the other hand, represent the most relevant class of analgesics. In the clinical setting anaesthetics and analgesics are delivered intravenously, intramuscularly, rectally or by inhalation, in the case of volatile agents. The pharmacologic effects achieved by the administered drug(s) depend on dosage, administration profile and patient sensitivity, amongst other factors.

In the 1950's Bickford (U.S. Pat. No. 2,690,178) and Bellville (U.S. Pat. No. 2,888,922) pioneered the design of novel dosing paradigms and administration procedures in anaesthesia health care. As the sophistication of monitoring systems and the understanding of bodily functions progressed, those early works were followed by several other contributions to the improvement of drug delivery. Zbinden and Westenskow, for example, proposed innovative methods to manage drug administration based on refined control algorithms (e.g. Westenskow et al., 1986 and other publications).

At present, the following dosing strategies are employed in the anaesthetic profession: manual bolus administration and/or continuous infusion, target controlled infusion (TCI), patient controlled sedation (PCS). Manual dosing performed by clinical caregivers exhibits a few drawbacks, including the necessity for the caregiver to select drug dosage on the basis of putative blood concentrations. Conversely, TCI enables the anaesthetist to target a predicted plasma or effect site concentration rather than selecting an infusion rate. The device controls drug delivery based on a built-in pharmacokinetic model of the specific drug. TCI does not account for two potential sources of error: the mismatch between predicted and actual concentrations; the pronounced inter- and intra-individual pharmacodynamic variability. The former cannot be evaluated and corrected intraoperatively. The latter can be compensated for by adjusting the targeted concentration to desired and/or undesired effects observed in the patient. That is to say, the appropriate targeted concentration can only be established after transient under- and/or overdosing. On the other hand, PCS devices can only be used in cooperative and adequately instructed patients. Their design leads to fluctuating levels of sedation and analgesia. PCS is regarded as a safe dosing strategy since an unresponsive patient is not able to operate the device. Drug administration ceases as a result. However, the time lag between dose delivery and maximal drug effect can lead to unintentional patient self-overdosing.

U.S. Pat. No. 6,745,764 (Hickle) discloses a drug delivery system with an integrated patient interface. The patient can operate the interface device to send dosage change requests to the drug delivery apparatus. The system is capable of determining patient responsiveness by evaluating the voluntary response of the patient to a stimulus (e.g., an auditory or vibratory command). Said system manages drug delivery in accord with patient interface requests and patient responsiveness data.

In recent years many have undertaken the challenge of improving anaesthesia health care. Some meaningful achievements in the design of administration procedures are summarized herein after.

European Pat. No. 1,547,631 (Barvais et al.) discloses a computer-aided system, which increases the safety of intravenous drug delivery and enables the transfer of expert knowledge to less experienced caregivers.

U.S. Pat. No. 6,807,965 (Hickle) discloses a system to conservatively manage drug delivery in accord with a safety data set, which is stored in a memory device. Said data set reflects both safe and undesirable physiological parameters and predefines normal ranges. The system monitors the physiological conditions of the patient, including the depth of patient unconsciousness. It compares the signals provided by patient monitors with the stored safety data set and responds by conservatively controlling (id est curtailing, limiting or ceasing) drug delivery.

P.C.T. Pat. No. WO 2001/083007 (Struys et al.) provides a system and method to run the administration of a drug based on a response profile of the patient. The patient's individual response profile is identified by means of a least-square algorithm. Following this approach, the differences between a sensed attribute of the patient and the estimated curve are minimized as to obtain the best fitting pharmacodynamic Hill curve. Use of an individualized Hill curve in adjusting drug delivery overcomes large pharmacodynamic variability amongst patients. In one embodiment of the invention, said system employs the patient's electroencephalographic (EEG) signal or the Bispectral Index (BIS™) to evaluate the depth-of-sedation response profile of the patient.

P.C.T. Pat. No. WO 2005/072792 (Struys et al.) discloses a care system, which manages the delivery of a medication in accord with a patient-specific response profile. The system determines the patient's response profile via the Bispectral Index and other processed EEG measures, such as the median frequency, the spectral edge and entropy metrics. Said system applies techniques from Bayesian statistics to adapt response profile parameters to changes occurring in the patient's response to the drug.

The problem of sedation is compounded by the lack of an artifact resistant respiration monitor for use during MAC and conscious sedation. Possible ways of monitoring patient respiration in the clinical setting include: visual inspection of the thorax; detection of thoracic electrical impedance changes; strain gauge measurement of thoracic circumference; ECG-derived detection of respiratory rate (e.g. respiratory sinus arrhythmia (RSA) detection, autoregressive spectral analysis of heart period, variations of electrical main axis of the heart and others); nasal thermistors; plethysmography; spirometry or airway pressure monitoring; capnographic appraisal of end-tidal CO2 partial pressure (PetCO2). Nevertheless, none of the aforementioned methods proves to be fully adequate for use during sedation. For example measuring the respiratory flow (or minute ventilation) through visual inspection is impractical and unreliable. If no other respiratory measurement is available, it represents the method anaesthetists rely on to verify whether the patient is apnoeic. It clearly performs very poorly in terms of reliability, precision, repeatability and automatization. Detecting thoracic impedance changes, on the other hand, allows for the assessment of tidal volumes, but the method is artifact prone. Movements of the patient and the cables reduce the SNR of the detected signal. Hence the measurements of respiratory rate which thoracic impedance changes yield are not suitable for intraoperative use. Capnography, the gold standard in respiration monitoring, suffers instead from false low readings, especially when upper airway obstruction occurs. The capnographic apparatus employs a nasal cannula for exhaled breath sampling. The cannula, 3 to 5 cm in length, does not impair spontaneous respiration; however it yields incorrect measurements if the patient breathes through the mouth. Another sampling setup makes use of a facial mask. The deadspace of the tubes and of the mask itself reduces the reliability of the PetCO2 measurements, therefore only the respiratory rate data are usually considered. Both set-ups are sensitive to shallow breathing and airway obstruction. To conclude, none of the ventilatory methods described so far meets the requirements for a fully satisfactory use in the clinical setting.

The adequacy of patient respiration can be indirectly determined by measuring transcutaneous CO2 tension (PtcCO2) and oxygen saturation (SpO2) (Akio et al., 2004). To this end, innovative devices combining pulse oximetry with transcutaneous CO2 sensing have recently entered the market. At present the following monitors are commercially available: V-Sign™ Sensor of SenTec AG (Therwil, Switzerland); TCM4, TCM40, TOSCA500 and MicroGas 7650 of Radiometer A/S (Copenhagen, Denmark). These devices employ a sensor positioned at the ear lobe for continuous measurement of patient heart rate, SpO2 and PtcCO2. Steady state bias and response time are satisfactory, therefore the sensors provide a fast and reliable respiratory indicator, which is suitable for use during MAC and conscious sedation.

P.C.T. Pat. No. WO 2002/041770 (Tschupp et al.) provides a sensor for measuring blood physiological parameters such as oxygen and carbon dioxide. Said sensor comprises of a measuring device and a digital sensor signal processor and provides a digital output signal.

P.C.T. Pat. No. WO 2005/110221 (Gisiger et al.) discloses a process to measure transcutaneous CO2 partial pressure at an ear lobe by means of a sensing device. The process employs a transcutaneous CO2 partial pressure measuring device and a heating system, which heats the sensor's contact surface.

In addition to its value as a patient well-being monitor, the PtcCO2 signal can serve as a surrogate endpoint for drug dosing. Here the underlying concept is derived from administration protocols used in cancer treatment. Drug dosing in oncology is frequently limited by the emergence of side effects rather than the achievement of the optimal therapeutic effect. This dosing paradigm is termed the “maximum tolerated systemic exposure” (MTSE). The application of the MTSE paradigm to MAC and conscious sedation implies the provision of analgesia and anxiolysis (the desired effects) by control of drug delivery based on respiratory inhibition (the adverse effect). To provide optimal analgesic or sedative treatment it is mostly not necessary to induce maximum tolerable respiratory depression (i.e. the systemic exposure), but rather the individualized optimal amount of respiratory depression will guide the treatment. Further on we will refer to this concept as IOSE (Individualized Optimal Systemic Exposure).

IOSE has a clear clinical value when the following conditions are met:

a) a simple and robust measure of the desired effect is not available;
b) the concentration-to-effect curves of the desired and undesired effects are related to each other.
Ad a). The desired effects of MAC and conscious sedation (analgesia, anxiolysis and/or sedation) can be easily measured in the awake and in the drowsy. Spontaneous complaints, movements, the VAS scale, and the OASS scale provide clear information whether the desired effects have been achieved. Nevertheless, these measures can be detected following a stimulus only. Undesired patient responses often lead to “overcorrections” by the anaesthetist, which in turn cause the occurrence of side effects. It has also been suggested that EEG-based indicators could provide a continuous measurement of the desired effects. For example, the Bispectral Index (BIS™) by Aspect Medical Systems is an EEG-derived parameter that evaluates the hypnotic component of anaesthesia independently of patient stimulation. However, the EEG shows wide fluctuations in moderately sedated patients and it is insensitive to opioids within the therapeutic range. To conclude, none of the methods discussed above can be used during MAC to estimate the desired effects.
Ad b). For mu-agonistic opioids and GABAergic drugs (such as benzodiazepines and propofol) the intensity of respiratory inhibition parallels that of analgesia and sedation. The interaction between drugs and nervous receptors explains this behaviour. Mu receptors in the brainstem and the thalamus mediate both the analgesic and the respiratory depressant effect of highly potent opioids. Benzodiazepines and propofol exert their sedative/hypnotic effect at GABA receptors, which have also been shown to cause respiratory inhibition. Therefore the drugs induce a respiratory depressant effect, which always correlates to analgosedation, and vice versa. The notion that drug binding to the identical receptor types causes both analgesia/sedation and respiratory depression implies that increasing concentrations of the drug(s) always leads to a concomitant increase of the analgesic/sedative and respiratory depressant effects. It is possible to “calibrate” analgesia/sedation to respiratory depression, the choice of a maximum tolerable degree of respiratory depression yields a maximum tolerable concentration, whose analgesic/sedative effect can immediately be determined. Based on e.g. the PCO2 dosing can be individualized, yielding the IOSE. Analgesia can only be assessed after a noxious stimulus has been applied. This proposed new dosing paradigm will enable the physician to obtain analgesic concentrations and individualize dosing using the most objective measurement of opioid effect in the therapeutic range: respiratory depression.

Under certain conditions (spontaneous respiration, modest pulmonary pathology), the IOSE concept can also be applied to intensive care unit (ICU) sedation. In this setting, conscious sedation is mainly delivered to provide tolerance of the endotracheal tube as well as postoperative/postinjury analgesia. All established methods to control ICU sedation are suboptimal. For example, measuring electrical CNS activity via BIS™ monitoring is not reliable at light sedation levels, and sedation scores cannot be obtained continuously. Nevertheless, there is a well established trend in ICU practice to move from controlled to assisted ventilation. This shift offers the possibility of using minute ventilation and/or respiratory gas measures (such as PCO2) as surrogate parameters for adequate sedation. In fact, oversedation results in decreasing minute ventilation and increasing PCO2; for undersedation the opposite applies. Therefore IOSE can be considered as a suitable dosing paradigm for intensive care analgosedation.

In the clinical setting anaesthetists can apply the IOSE concept by titrating drug delivery to the observed undesired effect since the measured endpoint is correlated to the therapeutic effect. For the sake of example, the care provider can manage drug administration by targeting a PCO2 of 50, 55 or 60 mmHg. The difference between the target PCO2 and the non-sedated value of about 40 mmHg accounts for the desired extent of pharmacologic effect. Moreover the IOSE paradigm can be embodied into an automated dosing device, as the present invention discloses.

The system herein disclosed provides an arrangement to control the administration of analgesics, sedative and/or hypnotics with respiratory depressant side effects. Drug delivery occurs to a spontaneously breathing patient who can be subject to drug-induced depression of consciousness. The drug delivery control apparatus takes into account monitored physiological conditions to determine the administration profile, which ensures adequate and safe sedation with minimal side effects. Respiratory impairment, blood deoxygenation and hypercapnic acidosis are the most significant adverse effects induced by the drug. The control apparatus manages drug delivery in accord with the feedback information provided by one or more patient monitoring devices. The feedback data reflect the respiratory state of the patient, including respiratory acidosis and the content of respiratory gases.

Herein before and after we refer to a feedback loop control system as understood in the field of automatic control. In engineering and mathematics, control theory deals with the behaviour of dynamical systems. A system where a transformation occurs or an action is performed is characterized by inputs and outputs. Feedback is the process whereby some proportion or, in general, a function of the output signal of the system is passed (“fed back”) to the input. This is often done intentionally, in order to control the dynamic behaviour of the system. Continuous feedback in a system generates a feedback loop.

In every feedback loop information about the result of a trans-formation or an action is sent back to the input of the system in the form of input data. Feedback leads to adjustments, which vary with the difference between actual input and desired input. Feedback loop control systems usually comprise:
a sensor of the variable to be controlled;
a reference input or setpoint that specifies the value the controlled variable should have;
a comparator that compares the actual sensed value, or feedback signal, with the setpoint or reference input. The output of the comparator is usually called an error signal, whose polarity determines which way a correction needs to be made;
a control mechanism or controller, which is activated by the error signal and manipulates the input of the system by means of an actuator to obtain the desired effect on the output of the system.

Several types of controllers or control mechanisms have been proposed, which differ in the inherent decision-making principle. A simple type of controller is a proportional controller. With this type of control, the controller output (that is, the control action) is proportional to the error signal. Proportional control is characterized by a very low degree of complexity but it has drawbacks, the most important being that for most systems it does not entirely remove the error, or deviance of the measured value from the desired value (setpoint). Alternatives to proportional control include proportional-integral (PI) control and proportional-integral-derivative (PID) control. These controllers can adjust process outputs based on the history (integral action) and the rate of change (derivative action) of the error signal, which increases the accuracy and stability of control. A more complex type of control is Model Predictive Control (MPC). An MPC controller relies on an empirical model of the dynamical system to predict the future behaviour of the dependent variables based on known values of the independent variables. MPC improves on simpler types of control by predicting how a system reacts to the inputs, that is, the effects produced by the inputs are known ahead of time. Feedback information is used to correct for model inaccuracies, since mathematical models often cannot completely describe system behaviour. Another advanced type of control is fuzzy logic control. In fuzzy logic the truth of any statement is a matter of degree. Fuzzy logic relies on mapping an input space to an output space by means of a set of rules, for example a list of “if-then” statements. Interpreting an “if-then” rule involves two distinct steps: evaluating the antecedent and applying that result to the consequent. Therefore a fuzzy logic controller is a controller that interprets the values of the inputs and, based on some set of rules, assigns values to the outputs.

As mentioned above, the error signal provides information on the magnitude and the direction of the adjustments made by the control mechanism on the input to the system. This means that a feedback loop control mechanism manipulates the input as to produce a decrease in the output if this exceeds the setpoint, and vice versa. The overall result is to stabilize the system and maintain the equilibrium around the desired setpoint even in the occurrence of disturbances. Applying this approach to a drug dosing paradigm, it implies that drug administration is adjusted depending on a condition sensed in the patient. Drug delivery is curtailed if the pharmacologic effect is too strong; it is increased if the effect is too weak. This can be understood as titrating drug delivery to effect. Generally speaking, the scope of a feedback controller goes well beyond that of an alarm or safety system which detects a dangerous condition for the patient and reduces or stops drug administration as a result.

Differently from feedback controllers, an open-loop controller is a type of controller, which computes its input into the system using only the current state and/or its model of the system. An open-loop controller (also called a non-feedback controller) does not use feedback to determine whether its input has achieved the setpoint. This means that the system does not observe the output of the process it is controlling. Consequently, a true open-loop system cannot compensate for disturbances in the system. Open-loop control principles have found a few applications in anaesthesia care, for example in TCI (Target Controlled Infusion) technology. One of the embodiments of the invention herein disclosed makes use of non-feedback control.

Unremitting vigilance, no fatigability, highly predictable and reproducible behaviour are general properties of machines which fully justify the design of an automated apparatus for drug delivery. These attributes are of special value in a dynamic situation, such as the delivery of anaesthesia, where lapses or delays might lead to catastrophic outcomes. Moreover, feedback devices are able to react adequately to the occurrence of painful stimuli and other sources of surgical disturbance by detecting a change in the measured endpoint. Pain, for example, triggers respiratory changes amongst other autonomic responses. Several studies report that pain perception has an overall excitatory influence on respiration (e.g. Sarton et al., 1997; Glynn et al., 1981). Such influence is exerted via the following physiological mechanisms:
pain intensifies carbon dioxide metabolism, that is, following a painful stimulation the rate of CO2 production in the body is increased. In fact, pain causes the release of catecholamines, which in turn intensify the cardiac and respiratory activity. Augmented physiological work determines an increase in both O2 consumption and CO2 production;
pain causes the physiological PCO2 setpoint in arterial blood to decline (Glynn et al., 1981).
the effect of pain is a chemoreceptor-independent tonic drive that increases minute ventilation, that is, the respiratory response to pain is not mediated by the central chemoreceptors in the medulla and the peripheral chemoreceptors of the carotid bodies, but rather centrally through the respiratory neurons within the brainstem (Sarton et al., 1997)
painful stimulation does not affect the slope of the CO2 response curve (representing minute ventilation versus PCO2 at different PO2 values).

The physiological mechanisms described above produce an overall increase in minute ventilation following painful stimulation. The effect on respiration reaches steady state in about 3 minutes and can be quantified as an approx. 20% increase in minute ventilation, producing in turn a decrease in carbon dioxide level.

Therefore, painful stimuli produce a change in respiratory conditions, which can be detected by the patient monitoring device and counteracted by the feedback design of the proposed system. Other surgical disturbances that affect patient respiration can be similarly governed by the system.

In addition, information on pharmacokinetics/pharmacodynamics (PK/PD) and on the dynamics of breathing and cardiovascular regulation can be implemented into our proposed system and method. In contrast, caregivers must learn the behaviour of the physiological system in the non-steady state and deal with inter-individual variability.

Finally, automated devices can be supplemented with safety overrides. For example, a minimal value of arterial oxygen saturation can be predefined to limit drug administration within a safe range. Other possible safety parameters are: the predicted drug concentration in the effect compartment or in blood; the total dose supplied to the patient; the administration rate. Safety overrides can either produce immediate cessation of drug delivery or maintain the existing drug concentration/administration rate.

Regardless of the specific setting, drug and endpoint, conscious sedation (frequently) and MAC (occasionally) are performed by caregivers who are not specially trained in the administration of anaesthetics, such as nurses. These caregivers are not familiar with the complexity of human respiratory control. Minute ventilation is in fact regulated by several endogenous and exogenous factors, including somatic/sensorial stimulation level, drug concentration, O2 and CO2 partial pressures. Each factor has a different influence on breathing in terms of both response magnitude and dynamics. Consequently, untrained caregivers are more prone to over- and underdose the patient than anaesthetists. In case of overdose, they are also less familiar with resuscitative measures. On the other hand, tasking trained anaesthetists with conscious sedation is expensive and often not feasible because of insufficient human resources. Delivering so-called “safe” drugs (low potent opioids like meperidine and midazolam) has limited clinical impact because it often leads to suboptimal, light sedation.

To conclude, the problem of sedation and analgesia is still unresolved. There is an evident clinical need of simplifying drug administration, increasing patient safety and comfort, and reducing clinical costs. The invention presented and disclosed here addresses these matters and offers a practical, effective and safe solution. The invented system and method prevent over- and underdosing while allowing the caregiver to provide analgosedation within safe limits. Other foci of the invention are discussed herein after.

A system for controlling administration of a respiratory depressant drug or mixture of drugs to a spontaneously breathing patient comprises a drug delivery unit, being adapted for indexed or continuous and automatic titration of a respiratory depressant drug or mixture of such drugs to said patient, and a control apparatus, receiving measurement signals relative to the respiratory state of said patient and issuing control signals to said drug delivery unit, wherein the control apparatus is adapted to keep said measurement signals relative to the respiratory state to a predetermined condition and thereby providing adequate sedation and/or analgesia to said patient.

The invention provides an apparatus and related methods for delivering one or more anaesthetic and/or analgesic drugs to a spontaneously breathing patient. The invention is directed towards alleviating the pain, discomfort and/or anxiety associated with medical or surgical treatments through the delivery of adequate and safe sedation. The invention is further directed towards optimizing drug administration, preventing patient under- and/or overdosing and minimizing the risks associated with respiratory depressant anaesthetics. The system is apt for use during medical or surgical treatments where relief of patient pain, discomfort and/or anxiety through patient sedation is desirable or required.

A care system in accordance with the invention comprises: one or more patient monitoring devices to monitor at least one physiologic condition reflecting the respiratory state in said patient; a drug delivery system to supply one or more drugs; a control apparatus to drive the delivery system.

The patient monitoring device provides one or more signals for the detection, measurement or inference of at least one physiologic condition reflecting said respiratory state in said patient. The drug delivery system supplies one or more drugs or mixtures of drugs to said patient. The control apparatus drives said drug delivery system and, in one form of the invention, interconnects said patient monitoring device and said drug delivery system.

The arrangement has the objective of controlling the administration of analgesics, sedative and/or hypnotics with respiratory depressant side effects to a spontaneously breathing patient who can be subject to some degree of drug-induced depression of consciousness. The drug delivery control apparatus takes into account monitored and/or inferred patient physiological conditions which reflect the respiratory state, including patient body contents of respiratory gases, to determine the optimal administration profile and ensure drug delivery within effective and safe ranges.

To accomplish the foregoing and other objectives, the procedure for managing patient pain and/or anxiety herein described includes the aspect of controlling the delivery of an anaesthetic agent based on predicted drug concentrations in the body. For example, drug administration can be based on the predicted drug concentration in the effect compartment or in blood. The prediction of anaesthetic concentrations is achieved via pharmacokinetic modeling and can take into account demographic covariates of the patient (such as age, sex, weight, height, and others).

In another aspect of the invention, the method takes into account the predicted pharmacologic side effects in order to determine the administration profile. Side effects are evaluated from the behaviour of a respiratory model built into the control system. For respiratory depressants such as opioids and propofol, respiratory impairment is the most visible undesired effect. In one embodiment, the method controls drug delivery based on respiratory indicators such as the respiratory rate (e.g. in non-intubated patients), minute ventilation (e.g. in intubated patients), tidal volumes. An extension of the method makes use of predicted body content of respiratory gases as an indirect appraisal of respiratory inhibition. Parameters reflecting the oxygen and carbon dioxide contents in the body, such as O2 saturation and CO2 partial pressure (or tension), are employed to determine the administration profile. In a preferred embodiment, the control apparatus makes use of predicted transcutaneous PCO2 values.

In accordance with one aspect of the invention, the administration profile is determined on the basis of a feedback signal provided by the patient monitoring device. The feedback signal reflects one or more physiological conditions, which allow the monitoring of the respiratory state and undergo modifications in presence of the anaesthetic. In one embodiment of the invention, the monitoring device provides information on patient respiration and/or respiratory acidosis and/or on body content of respiratory gases following the administration of respiratory depressants. In a preferred embodiment, the drug delivery control apparatus receives data from a combined pulse oximetry/transcutaneous PCO2 sensor.

In another aspect of the invention, the method uses the information inherent in the feedback signal to improve the safety of drug delivery. The control apparatus continuously redetermines the administration profile based on measured respiratory indicators with the combined objective of achieving the required therapeutic effect and preventing drug overdosing. If the impairment of the respiratory drive becomes pronounced, the system limits or ceases drug delivery to minimize patient endangerment.

In yet another aspect of the invention, the feedback signal provided by the monitoring device is used to tune the behaviour of the underlying respiratory model to the responsiveness of the patient. In case of signal loss and/or sensor malfunction, failure or disconnection, the control system can switch from closed-loop to open-loop operation and continue providing effective pain and/or anxiety management through adequate drug delivery.

An advantage of the method herein described is that it can tailor drug administration to the specific needs of the patient, depending on the stimulation level, the desired analgesic and anxiolytic effect, the individual sensitivity to the drug. Regardless of the clinical setting, drug and endpoint, the method ensures patient safety and comfort, minimizes the risk of drug under- and overdosing, and reduces clinical costs.

Further features and advantages of the invention and the structure and operation of various embodiments of the invention are described herein after.

### SHORT DESCRIPTION OF THE DRAWINGS

FIG. 1 Basic conceptual schematic drawing of a system according to one embodiment of the invention,

FIG. 2 Diagram of effect and side effect vs. drug concentration for the opioid remifentanil,

FIG. 3 Diagram of effect and side effect vs. drug concentration for the opioid alfentanil,

FIG. 4 Diagram of effect and side effect vs. drug concentration for propofol.

### DESCRIPTION OF PREFERRED EMBODIMENTS

The embodiments described here after are not intended to be exhaustive or to limit the invention to the exact form as shown. The embodiments were selected to explain the principles of the invention and its application.

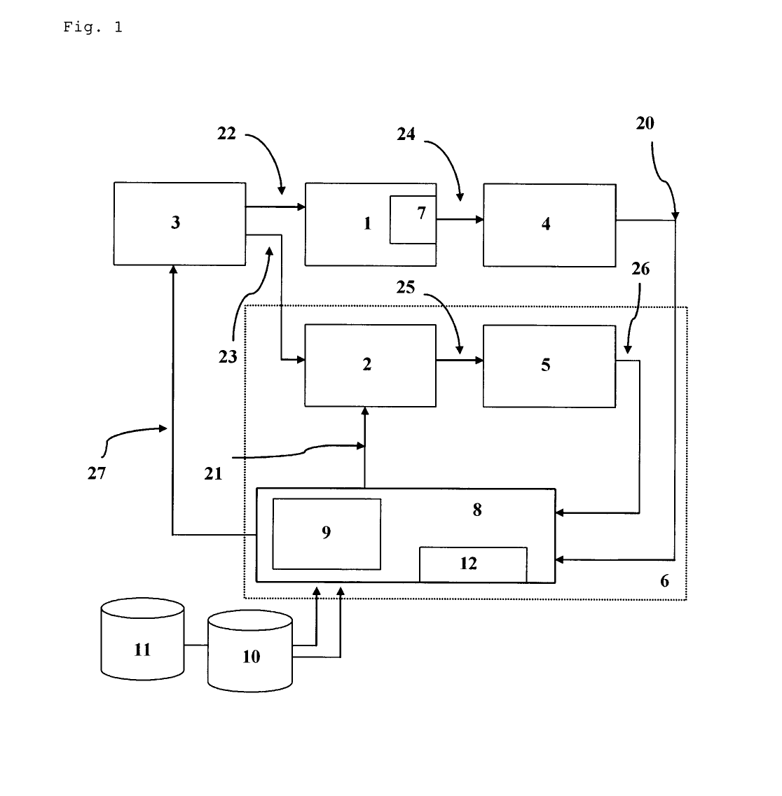

FIG. 1 shows the preferred basic structure of a device according to an embodiment of this invention, providing analgesics, sedatives and/or hypnotics with respiratory depressant side effects to a spontaneously breathing patient 1 undergoing a medical or surgical procedure. The block 1, although mentioned as and designating the patient, comprises at least one sensor 7, providing an output signal 24, being a sensory signal relative to the respiratory state of the patient. Of course, there can be more than one sensor and these sensors can provide signals for different indicators of said respiratory state. Of course, the patient 1 has no input signal as such. However, FIG. 1 shows that the device comprises a drug delivery unit 3 having as output an indexed or continuous output of analgesics, sedatives and/or hypnotics, to be applied to the patient via a drug delivery means 22 (e.g. an infusion line). Furthermore, an electronic representation of the actual output of analgesics, sedatives and/or hypnotics from the drug delivery unit 3 is submitted via 23 as an entry signal to a processor unit 2 (inside control apparatus 6), representing a database and computer software product within a processor unit for a Patient Model. A Patient Monitor Device 4 is measuring the respiratory state of the patient 1 through the input signals provided by one or more of the sensors 7. A Model Prediction Calculator 5 (inside control apparatus 6) is inferring respiratory state from the Patient Model 2 received via an electronic representation 25 and a control unit 8 (inside control apparatus 6) issuing electronic dosing communication 27 to the device delivery unit 3 for indexed or continuous titration. Said drug delivery is according to the respiratory state based on direct patient measurements 20 (closed-loop mode, one of the preferred embodiments) or on model predictions 26 (open-loop mode, one of the preferred embodiments, especially in case of loss of sensor signal and target controlled infusion mode). The said titration by the control unit 8 comprising a control mechanism running on a processor unit with a decision-making principle such as PID (proportional-integral-derivative) implemented in software is guided by the error signal calculated by comparing setpoint and patient measurement 20 or model prediction 26. The decision-making principle of the control mechanism of this embodiment is not restricted to PID but can be based on other suitable principles. During closed-loop operation the Patient Model 2 is adapted and updated in a continuous or indexed form via 21 based on actual patient measurements 20.

In a preferred embodiment the control unit 8 contains a Display 9 and one or more Input-Output Systems 12 for user interaction. A preferred embodiment furthermore comprises a Drug Database 10 containing pharmacokinetic and pharmacodynamic properties of drugs or mixtures of drugs to be administered to the patient, especially enabling presetting of the control unit 8 and the patient model 2 inside the control apparatus 6. The pharmacodynamic data includes not only properties for the therapeutic effect but also data about the side effects, particularly with regard to the impact on dynamics of breathing and cardiovascular regulation. In this preferred embodiment such data from the said Drug Database 10 is loaded into the control apparatus 6 for subsequent use.

In a preferred embodiment the Control Apparatus 6 is supplemented with additional safety overrides consisting of a) a minimal tolerable arterial oxygen saturation as a “hard” safety override with immediate cessation of drug administration in case of violation, b) a user presetable maximum and minimum predicted drug concentration in the effect compartment as a “soft” safety override at which the concentration will be maintained when reached, resulting in an open loop mode of operation and notification of the user and c) limits on infusion rates (in case of i.v. drugs), total drug dose and lock-out times for drug administration. In a preferred embodiment all safety information and constraints are stored in a safety database 11 which is attached to the control apparatus 6.

In a preferred embodiment the respiratory state of the patient is evaluated by appraisal of the respiratory acidosis. The respiratory acidosis is preferably measured by quantifying carbon dioxide content in blood, or in another preferred embodiment by quantifying the pH in blood. This can be done in various ways and the information is obtained as such to deliver an indication relative to the respiratory state of the patient.

In a preferred embodiment the carbon dioxide content in the blood of the patient is measured by a fast transcutaneous carbon dioxide partial pressure measurement. In another embodiment the carbon dioxide content in the blood of the patient is measured by end-tidal measurement of carbon dioxide. All such measurement systems are preferably complemented with an independent sensor for respiratory rate for the detection of a single fault condition in the sensory system.

For analgesia the preferred drugs are opioids, specifically alfentanil, fentanyl, remifentanil and sufentanil.

A mixture of respiratory depressant drugs with non respiratory depressant analgesic or sedative drugs is preferred in patient cases where atypical respiratory depressant drug and/or carbon dioxide sensitivity is observed. In a preferred embodiment the non respiratory depressant drug is ketamine.

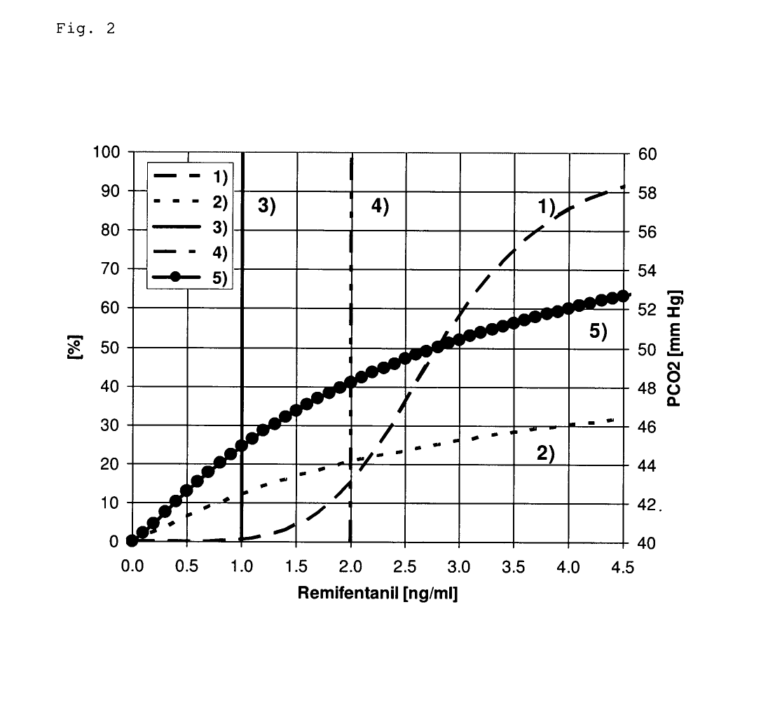

FIG. 2 shows a diagram of effect and side effect vs. drug concentration for the preferred use of the opioid remifentanil for analgesia. Remifentanil is used since the mid 1990's, predominantly for intraoperative analgesia and conscious sedation. Available data describe its analgesic and respiratory depressant potency (target concentrations aimed for by the individual using patient controlled analgesia (Schraag et al., 1998; Cortinez et al., 2005) and entire concentration effect curves based on multiple observations within individuals for respiratory depression, with measured PaCO2 for respiratory depression capture (Bouillon et al., 2003). The diagram shown in FIG. 2 has a) on the left ordinate percentage of specific maximum effect as described below, b) on the right ordinate partial CO2 pressure in [mm Hg] and c) on the abscissa concentration of remifentanil in [ng/ml]. The legend to the data traces of the diagram in FIG. 2 is as follows: 1) On left ordinate: analgesia measured as percentage of patients experiencing adequate pain relief (visual analogue scale<3 with 0 denoting no pain, 10 worst imaginable pain). A C50 (concentration required to achieve half of the effect) of 2.8 ng/ml was reported, slope extrapolated. Data was obtained during extracorporeal shock wave lithotripsy (ESWL) in an immersion lithotripter. 2) Also on left ordinate: respiratory depression measured as percent decrease of minute ventilation at uncontrolled PCO2 (clinical situation; note that the accompanying hypercarbia partially antagonizes the effect of the opioid on minute ventilation). 3) Fixed value expressed as perpendicular line on abscissa: C50 for respiratory depression, i.e. the remifentanil concentration at the effect site leading to a 50% decrease of isohypercapnic minute ventilation. Note that this value can be used to predict both analgesia and minute ventilation at uncontrolled PCO2, e.g. 1 C50 for respiratory depression yields substantial pain relief (postsurgical pain, abdominal procedures) and minimally depresses resting ventilation. 4) Fixed value expressed as perpendicular line on abscissa: median concentration measured in cardiac surgery and orthopedic patients operating a patient controlled analgesia device to achieve adequate postoperative pain relief. 5) On right ordinate: PCO2 expressed as absolute value in mmHg.

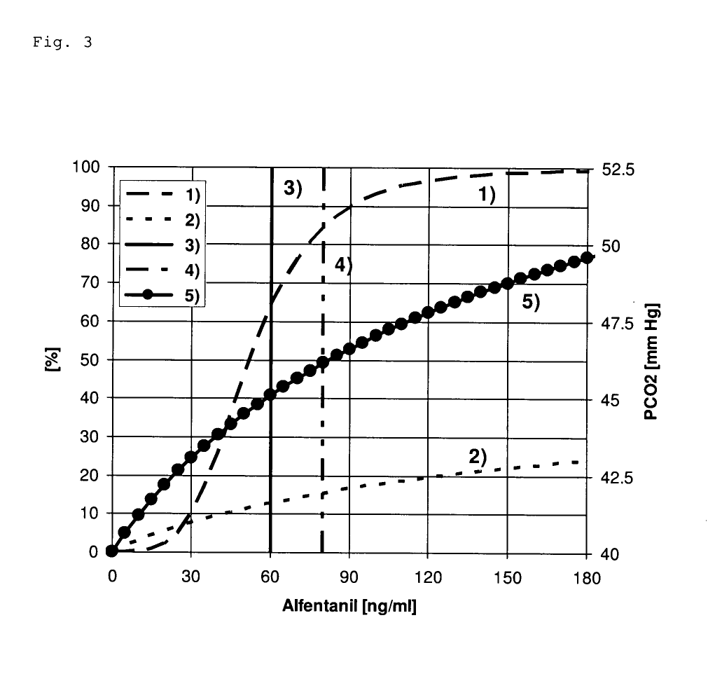

FIG. 3 shows a diagram of effect and side effect vs. drug concentration for the preferred use of the opioid alfentanil for analgesia. Alfentanil is used since the late 1980s for both intra- and postoperative analgesia. Available data describe its analgesic and respiratory depressant potency. Target concentrations aimed for by the individual using patient controlled analgesia (van den Nieuwenhuyzen et al., 1997; Schraag et al., 1999) and entire concentration effect curves based on multiple observations within individuals for respiratory depression with PaCO2 to capture respiratory depression (Bouillon et al., 1999) are available. The diagram shown in FIG. 3 has a) on the left ordinate percentage of specific maximum effect as described below, b) n the right ordinate partial CO2 pressure in [mm Hg] and c) on the abscissa concentration of alfentanil in [ng/ml]. The legend to the data traces of the diagram in FIG. 3 is as follows: 1) On left ordinate: analgesia measured as percentage of patients experiencing adequate pain relief (visual analogue scale<3 with 0 denoting no pain, 10 worst imaginable pain). A C50 of ng/ml was reported, slope extrapolated, based on cases in general surgery, gynaecological and orthopedic procedures. 2) Also on left ordinate: respiratory depression measured as percent decrease of minute ventilation at uncontrolled PCO2 (clinical situation; note that the accompanying hypercarbia partially antagonizes the effect of opioid on minute ventilation). 3) Fixed value expressed as perpendicular line on abscissa: C50 for respiratory depression, i.e. the alfentanil concentration at the effect site leading to a 50% decrease of isohypercapnic minute ventilation. Note that this value can be used to predict both analgesia and minute ventilation at uncontrolled PCO2, e.g. 1 C50 for respiratory depression yields substantial pain relief (postsurgical pain, abdominal procedures) and minimally depresses resting ventilation. 4) Fixed value expressed as perpendicular line on abscissa: median concentration measured in post-cardiac surgery patients operating a patient controlled analgesia device to achieve adequate pain relief, fixed value expressed as perpendicular line on abscissa. 5) On right ordinate: PCO2 expressed as absolute value in mmHg.

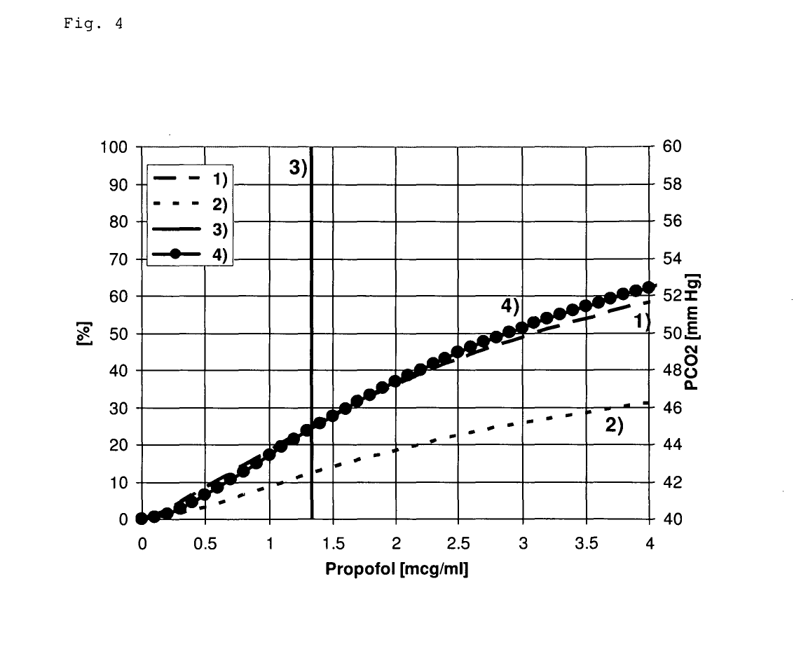

FIG. 4 shows a diagram of effect and side effect vs. drug concentration for the preferred use of propofol for sedation. Propofol is used since the late 1980's, both for conscious sedation and provision of anaesthesia. Available data describe its sedative (e.g. suppression of the Bispectral Index, an EEG derived parameter (Bouillon et al., 2004, “Pharmacodynamic interaction . . . ”)) and respiratory depressant potency (Bouillon et al., 2004, “Mixed-effects modeling . . . ”). For these endpoints entire concentration effect curves are available with PaCO2 to capture respiratory depression. The diagram shown in FIG. 4 has a) on the left ordinate percentage of specific maximum effect as described below, b) on the right ordinate partial CO2 pressure in [mm Hg] and c) on the abscissa concentration of propofol in [μg/ml]. The legend to the data traces of the diagram in FIG. 4 is as follows: 1) On left ordinate: hypnosis measured as decrease of the Bispectral index (BIST™), an EEG derived surrogate endpoint for hypnosis/sedation. A value of 50 corresponds to surgical anaesthesia, 60-75 is adequate for conscious sedation. 2) Also on left ordinate: respiratory depression measured as percent decrease of minute ventilation at uncontrolled PCO2 (clinical situation; note that the accompanying hypercarbia partially antagonizes the effect of propofol on minute ventilation). 3) Fixed value expressed as perpendicular line on abscissa: C50 for respiratory depression, i.e. the propofol concentration at the effect site leading to a 50% decrease of isohypercapnic minute ventilation. Note that this value can be used to predict both sedation and minute ventilation at uncontrolled PCO2, e.g. 1 C50 for respiratory depression yields adequate sedation and minimally depresses resting ventilation. 4) On right ordinate: PCO2 expressed as absolute value in mmHg.

According to FIGS. 2 to 4 and considering additional statistical variance the setpoint for carbon dioxide level for therapeutic effect in a preferred embodiment ranges from 35 to 80 mmHg, preferably in a range of 45 to 65 mmHg and ideally in a range of 48 to 55 mmHg partial pressure of carbon dioxide. These ranges of values are especially useful for transcutaneous acquisition of the carbon dioxide level. However, it is also possible to measure CO2 directly as a gas near the lungs of the patient, even within an intubation of the patient. The values to be chosen can be adjusted by the care provider.

For anaesthesia cases with moderate to elevated pain levels as experienced from minor to major surgery the preferred drug is a mixture of propofol and remifentanil. For fastest recovery and best therapeutic results the dosing ratio between remifentanil and propofol is preferably chosen such as to maintain a fixed concentration range in plasma or effect site in the range of 0.0015:1 and 0.0035:1. For sedation cases with low to moderate pain levels as experienced in minor surgery or diagnostic interventions that require an additional relief from discomfort and/or anxiety the preferred ratio between remifentanil and propofol is in the range of 0.0002:1 and 0.0008:1.

The control apparatus is adapted to keep said measurement signals relative to the respiratory state to a predetermined condition and thereby providing adequate sedation and/or analgesia to said patient. This means, that preferably the database 10 or a user chooses a preset setpoint as mentioned above, e.g. the carbon dioxide level. This condition can be a given point on a control curve, which is controlled with usual closed-loop control systems, where a feedback control monitors the system. Such closed-loop control systems known from control theory can also use intervals with lower and upper levels, e.g. of the carbon dioxide level. Such conditions are meant to be achieved with the control apparatus 6 in connection with the further elements as e.g. shown in FIG. 1.

In a preferred embodiment the said continuous system mode is implemented as discrete time based system or indexed system using time constants that are in relation to but preferably lower than the underlying physiological system time constants. For the said drugs and mixtures of drugs and the changes of the respiratory state of a patient induced by said drugs a preferred range of system time constant is 1 to 60 seconds. Consequently a system update rate (e.g system internal states update, drug delivery unit update, patient monitor read-out) is preferably chosen in the same range of 1 to 60 seconds, e.g. with 5 seconds intervals, 10 seconds intervals or 20 seconds intervals.

In a preferred embodiment of the open-loop system the said model predictions are used for target controlled infusion (TCI, an infusion method to reach a preset concentration as fast as possible without overshoot). This means that, according to FIG. 1, the Patient Monitor Device 4 and therefore measurements 20 are a) not used or they are not provided within one form of this embodiment or b) they are used as an input value in another form of this embodiment. In the first form of this embodiment the level of predicted respiratory depression expressed as fractional respiration of baseline respiration is used to limit the infusion rate that is calculated to reach a preset plasma or effect site concentration. In the second form of this embodiment the actual measurements (20) relative to the respiratory state of the patient are used to calculate the fractional respiration by means of the respiratory model. This data is then used to limit the infusion rate to achieve a preset plasma or effect site concentration. Therefore in both forms of this preferred embodiment the maximal accepted reduction of fractional respiration represents an infusion rate limiting factor in the TCI method and is selectable by the care provider. In a preferred embodiment the said fractional respiration is in a range of 0.4 to 0.95 and ideally in the range of 0.6 to 0.8 of baseline respiration. The combination of a) the pharmacokinetic model of a respiratory depressant drug for the TCI algorithm with b) its pharmacodynamic effect on respiration and with c) the respiratory model with carbon dioxide kinetics and dynamics in these embodiments ensures that the preselected concentrations are reached as fast as possible while maintaining respiration at safe levels.

### REFERENCE NUMERALS

1 patient
2 patient model processor
3 drug delivery unit
4 patient monitor device
5 model prediction calculator
6 control apparatus (dotted box)
7 sensor unit
8 control unit
9 display
10 drug database
11 safety database
12 Input-Output Systems
20 patient measurements
21 patient model update
22 drug delivery to patient (e.g. infusion line)
23 electronic representation of drug delivery
24 sensory signals
25 electronic model representation
26 electronic representation of model predictions
27 electronic dosing communication

1. A system for controlling administration of a respiratory depressant drug or mixture of drugs to a spontaneously breathing patient, comprising:
a drug delivery unit, being adapted for indexed or continuous and automatic titration of a respiratory depressant drug or mixture of such drugs to said patient, and
a control apparatus, receiving measurement signals relative to the respiratory state of said patient and issuing control signals to said drug delivery unit,
wherein the control apparatus is adapted to keep said measurement signals relative to the respiratory state to a predetermined condition and thereby providing adequate sedation and/or analgesia to said patient.

2. The system according to claim 1, wherein the control apparatus achieves selectable drug plasma or effect site concentrations via said drug delivery unit as fast as possible while limiting the drug dosing rate to ensure a fractional respiration in a preferred range of 0.4 to 0.95 and ideally in a range of 0.6 to 0.8 at all times.

3. The system according to claim 1, wherein said measurement of the respiratory state is by appraisal of the respiratory acidosis via a carbon dioxide content measurement in blood.

4. The system according to claim 3, wherein said condition lies in the range of 35 to 80 mmHg, preferably in the range of 45 to 65 mmHg and ideally in the range of 48 to 55 mmHg partial pressure of carbon dioxide.

5. The system according to claim 3, wherein said carbon dioxide measurement is an end-tidal partial pressure measurement.

6. The system according to claim 3, wherein said carbon dioxide measurement is a transcutaneous partial pressure measurement.

7. The system according to claim 1, where said measurement of the respiratory state is by appraisal of the respiratory acidosis via a pH measurement in blood.

8. The system according to claim 1, wherein said control apparatus contains a pharmacokinetic and pharmacodynamic model of the drug or mixture of drugs to be used by the delivery unit.

9. The system according to claim 1, wherein said control apparatus further contains a model for the drug induced effect on the respiratory state and a respiratory model including the kinetics and dynamics of carbon dioxide as indicator of the respiratory acidosis of said patient.

10. The system according to claim 1 wherein said drug delivery unit comprises an infusion and/or syringe pump.

11. The system according to claim 1, wherein said continuous titration is time discrete and based on the system update rate, especially with time intervals ranging from 1 to 60 seconds.

12. The system according to claim 1, wherein said drug or mixture of drugs delivered by said drug delivery unit is or comprises an opioid, especially wherein said opioid is remifentanil, alfentanil, sufentanil or fentanyl.

13. The system according to claim 1, wherein said drug or mixture of drugs delivered by said drug delivery unit is or comprises propofol.

14. The system according to claim 1, wherein said mixture of drugs contains at least one respiratory depressant drug and additionally non respiratory depressant sedative or analgesic drugs, especially wherein said mixture of drugs contains an opioid and ketamine.

15. A mixture of drugs containing remifentanil and propofol in a ratio to achieve a steady state concentration ratio in plasma or at the effect site, for the use in anaesthesia, between 0.0015:1 and 0.0035:1, or, for the use in sedation, between 0.0002:1 and 0.0008:1, especially to be used in connection with a system according to claim 1.

16. A method for controlling administration of a respiratory depressant drug or mixture of drugs to a spontaneously breathing patient, comprising the steps of obtaining measurement signals relative to the respiratory state of said patient, issuing control signals to a drug delivery unit, being adapted for indexed or continuous and automatic titration of a respiratory depressant drug or mixture of such drugs to said patient, wherein the control apparatus is adapted to keep said measurement signals relative to the respiratory state to a predetermined condition and thereby providing adequate sedation and/or analgesia to said patient.
